(19) United States
(12) Patent Application Publication
(10) Pub. No.: US 2003/0018061 A1
(43) Pub. Date: Jan. 23, 2003

(54) Novel remedies with the use of beta 3 agonist

(76) Inventors: Kohei Ogawa, Shizuoka (JP); Hiroshi Umeno, Shizuoka (JP)

Correspondence Address: YOUNG & THOMPSON, 745 SOUTH 23RD STREET 2ND FLOOR, ARLINGTON, VA, 22202

(21) Appl. No.: 10/182,375

(22) PCT Filed: Jan. 26, 2001
(86) PCT No.: PCT/JP01/00553

(30) Foreign Application Priority Data
Jan. 28, 2000 (JP) 2000-20733

(51) Int. Cl. A61K 31/403; A61K 31/381; A61K 31/343

(52) U.S. Cl. 514/411; 514/468; 514/443

(57) ABSTRACT

Provided is a therapeutic agent comprising at least one member selected from the group consisting of an anticholinergic agent, a monoamine reuptake inhibitor, a lipase inhibitor, a selective serotonin reuptake inhibitor, insulin, an insulin secretagogue, biguanide, an α-glucosidase inhibitor, an insulin resistance improving agent, a HMG-CoA reductase inhibitor, an anion exchange resin, a clofibrate type drug and a nicotinic acid type drug, and a compound having a β3 agonist activity. The β3-agonist has an activity of inhibiting dysuria. Further, when used together with a remedy for dysuria such as propiverine, oxybutynin hydrochloride or tolterodine, it exerts an enhanced anti-dysuria effect. When used together with an antiobestic agent such as sibutramine or orlistat, it exerts an enhanced antiobestic effect. When used together with an antidiabetic agent such as insulin, glibenclamide, acarbose or rosiglitazone, it exerts an enhanced antidiabetic effect. When used together with an antilipemic agent such as bezafibrate or pravastatin, it exerts an enhanced antilipemic effect.

### FIELD OF THE INVENTION

The present invention relates to novel therapeutic agents that use a β3 agonist.

### BACKGROUND OF THE INVENTION

β adrenaline receptors are classified into β1, β2, and β3. It is considered that β1 stimulation increases the pulse rate, β2 stimulation induces relaxation of smooth muscle tissue and reduces the blood pressure, and β3 promotes lipolysis of adipose cells and increases thermogenesis. Accordingly, it is shown that a β3 agonist is useful as a therapeutic agent for diabetes, obesity and prevention of hyperlipidemia (Nature 309, p163-165 (1984); Int. J. Obes. Relat. Metab. Disord. 20, p191-199 (1996); Drug Development Research 32, p69-76 (1994); J. Clin. Invest. 101, p2387-2393 (1998)).

Recently, it has been shown that β adrenaline receptors are expressed in the detrusor muscle, and that the detrusor muscle relaxes with a β3-agonist (J. Urinol. 161, p680-685 (1999); J. Pharmacol. Exp. Ther. 288, p1367-1373 (1999)). On the other hand, while flavoxate hydrochloride, oxybutynin hydrochloride, propiverine hydrochloride and tolterodine have been used in treatment of patients affected by pollakiuria or incontinence of urine up to now (Folia Pharmacologica Japonica, Vol. 113, p157-166 (1999); Eur. J. Pharmaco. 349, p285-292 (1998)), their side effects include mouth dryness, difficulty in urinating, and constipation (RINSHOU HINYOUKIKA, Vol. 52, p277-285 (1998)), and the situation can not be considered satisfactory.

Further, while sibutramine is used as an antiobestic agent, its side effects have been reported to include elevation of blood pressure, mouth dryness and constipation (Int. J. Obesity, 21, S30-36 (1997)). Sibutramine is a monoamine reuptake inhibitor, and examples of other drugs belonging to this group include milnacipran, duloxetine, and venlafaxine (Medicine and Drug Journal, Vol. 36, No. 2, p151-157 (2000)). In addition, fluoxetine (Am. J. Clin. Nutr. 64, p267-273 (1996)), sertraline (J. Endocrinol. Invest. 19, p727-733 (1996)), paroxetine (Drugs 55, p85-120 (1998)) and fluvoxamine (J. Clin. Psychiatry 57, p346-348 (1996)) are considered useful as antiobestic agents. Further, orlistat has also begun to be used as an antiobestic agent, however, although the number of cases is small, gastrointestinal dysfunction side effects (fatty stool, defecation increase, abdominal pain, and impending defecation desire) have been reported (J. Clin. Pharmacology 37, p453-473 (1997)).

As therapeutic agents for type II diabetes mellitus, insulin, an insulin secretagogue, a biguanide, an α-glucosidase inhibitor, and an insulin resistance improving agent are used. As compounds having an effect that promotes insulin secretion, glibenclamide, glipizide, gliclazide, glimepiride, tolazamide, tolbutamide, acetohexamide, chlorpropamide, glyclopyramide, and meglitinide are used. Further, compounds called repaglinide and nateglinide have begun to be used, while compounds in clinical testing include mitiglinide (NIPPONRINSHO (an extra issue), Diabetes Mellitus 2, p147-185 (1997)); IGAKU NO AYUMI, Diabetes Mellitus, Vol. 188, p491-495 (1999)). However, these insulin secretagogues have side effects of appetite promotion and hypoglycemia, and the situation is not necessarily a satisfactory one. As biguanide, metformin and buformin are used, and they are known to cause lactic acidosis (IGAKU NO AYUMI, Diabetes Mellitus, Vol. 188, p504-509 (1999)). As α-glucosidase inhibitors, acarbose (Ann. Pharmacother. 30, p1255-1262 (1996) and voglibose (NIPPONRINSHO, Vol. 55 (Suppl.), p114-119 (1997)) are used clinically, and miglitol (Pharmacology, 43, p318-328 (1991)) and emiglitate (Eur. J. Clin. Pharmacol. 41, p561-567 (1991)) are in clinical testing. These α-glucosidase inhibitors have side effects of abdominal distension, flatus increase, loose passage and diarrhea (IGAKU NO AYUMI, Diabetes Mellitus, Vol. 188, p496-499 (1999)). As insulin resistance improving agents, the thiazolidinedione derivatives troglitazone, peoglitazone and rhodiglitazone (Japanese Patent Application Laying-Open (kokai) No. 55-22636; Japanese Patent Application Laying-Open (kokai) No. 60-51189; Japanese Patent Application Laying-Open (kokai) 6-157522; European Patent No. 0306228; Diabetes 37, p1549-1558 (1988); Diabetes 43, p1203-1210 (1994); Diabetes 41, p476-483 (1992); IGAKU NO AYUMI, Diabetes Mellitus, Vol. 188, p500-503 (1999)) are used clinically. Further, compounds in clinical testing include MCC-555 (Br. J. Pharmacol. 125, p767-770 (1998)), GI-262570 (WO97/31907), JTT-501 (Diabetologia 42, p151-159 (1999)), and KRP-297 (Diabetes 47, p1841-1847 (1998)). A side effect of insulin resistance improving agents is weight gain, and for some insulin resistance improving agents a side effect of fulminant hepatitis has been cited although the frequency is low (IGAKU NO AYUMI, Diabetes Mellitus, Vol. 188, p500-503 (1999)).

Antilipemic drugs include HMG-CoA reductase inhibitors, anion exchange resins, clofibrate type drugs and nicotinic acid type drugs. As HMG-CoA reductase inhibitors, pravastatin (YAKUGAKU JOURNAL, Vol. 111, p469-487 (1991); U.S. Pat. No. 4346227), simvastatin (Atherosclerosis 101, p117-125 (1993); U.S. Pat. No. 4,444,784), fluvastatin (Journal of clinical therapeutics & medicine, Vol. II (Suppl. 1), p153-180 (1995)); atorvastatin (Am. J. Cardiol. 79, p1248-1252 (1997)), and cerivastatin (Atherosclerosis, 135, p119-130 (1997)) are used clinically, and nisvastatin (Life Sci. 65, p1493-1502 (1999)) and S-4522 (Bioorg. Med. Chem. 5, p437-444 (1997)) are in clinical testing. Side effects of HMG-CoA reductase inhibitors include gastrointestinal dysfunction, liver enzyme increase, CPK increase, and myopathy (Mol. Med. 31, p544-549 (1994)). Anion exchange resins include cholestyramine (KISO TO RINSHOU (The clinical report), Vol. 16, p150-169 (1982)) and cholesthimide (Journal of clinical therapeutics & medicine, Vol. 12, p1263-1304 (1996)), and their side effects include a bloating sensation, constipation, hard stool and hepatotoxicity. Clofibrate-base drugs include clofibrate, simfibrate, clinofibrate, bezafibrate, fenofibrate, ciprofibrate and gemfibrozil, and their side effects include gastrointestinal dysfunction, hepatopathy, gallstone, lowering of libido, CPK increase and myopathy. Nicotinic acid-base drugs include nicotinic acid, nicomol, niceritrol and tocopherol nicotinate, and their side effects include a heat sensation, blushing, mouth dryness, lowering of glucose tolerance, gastrointestinal dysfunction and hyperuricemia (Mol. Med. 31, p544-549 (1994)). In addition, combined use of clofibrate type drugs and statine-base compounds is a contraindication because of hepatotoxicity.

As described above, the situation regarding these drugs is not yet satisfactory. The provision of a novel, useful and superior remedy than can be used in treatment and prevention of dysuria, obesity, diabetes mellitus, hyperlipidemia and the like has been long awaited.

### DISCLOSURE OF THE INVENTION

In order to solve the above problems, the present inventors have confirmed that β3-agonist has an activity that inhibits dysuria. Further, we discovered that when used together with a therapeutic agent for dysuria such as propiverine, oxybutynin hydrochloride or tolterodine, the β3-agonist exerts an enhanced antidysuric effect, when used together with an antiobestic agent such as sibutramine or orlistat, it exerts an enhanced antiobestic effect, when used together with an antidiabetic agent such as insulin, glibenclamide, acarbose or rosiglitazone, it exerts an enhanced antidiabetic effect, and when used together with an antilipemic agent such as bezafibrate or pravastatin, it exerts an enhanced antilipemic effect. Thus, we succeeded in completing the present invention.

That is, the present invention provides a therapeutic agent characterized by containing at least one member selected from the group consisting of an anticholinergic agent, a monoamine reuptake inhibitor, a lipase inhibitor, a selective serotonin reuptake inhibitor, insulin, an insulin secretagogue, biguanide, an α-glucosidase inhibitor, an insulin resistance improving agent, a HMG-CoA reductase inhibitor, an anion exchange resin, a clofibrate type drug and a nicotinic acid type drug, and a compound having a β3 agonist activity, or a treatment method characterized by administration of the therapeutic agent.

More specifically, the present invention provides a therapeutic agent for pollakiuria and incontinence of urine characterized by comprising at least an anticholinergic agent and a compound having a β3 agonist activity, as well as a treatment method for pollakiuria and incontinence of urine characterized by administration of the therapeutic agent.

Further, the present invention provides a therapeutic agent for obesity characterized by comprising at least one member selected from the group consisting of a monoamine reuptake inhibitor, a lipase inhibitor and a selective serotonin reuptake inhibitor, and a compound having a β3 agonist activity, as well as a treatment method for obesity characterized by administration of the therapeutic agent.

In addition, the present invention provides a therapeutic agent for diabetes mellitus characterized by comprising at least one member selected from the group consisting of insulin, an insulin secretagogue, biguanide, an α-glucosidase inhibitor and an insulin resistance improving agent, and a compound having a β3 agonist activity, as well as a treatment method for diabetes mellitus characterized by administration of the therapeutic agent.

Further, the present invention provides a therapeutic agent for hyperlipemia characterized by comprising at least one member selected from the group consisting of a HMG-CoA reductase inhibitor, an anion exchange resin, a clofibrate type drug and a nicotinic acid type drug, and a compound having a β3 agonist activity, as well as a treatment method for hyperlipemia characterized by administration of the therapeutic agent.

This specification includes part or all of the contents as disclosed in the specification of Japanese Patent Application No. 2000-20733, which is a priority document of the present application.

### PREFERRED EMBODIMENT OF THE INVENTION

Preferably, the β3-agonist of the present invention is, for example, a compound shown by any one of the following general formula (I), general formula (II), general formula (III) and a salt thereof.

A compound of the formula (I):

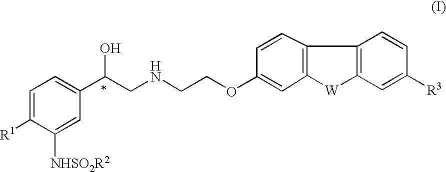

[wherein, R¹ represents a hydrogen atom, a halogen atom or a hydroxyl group, and R² represents a lower alkyl group or a benzyl group. R³ represents OR, a halogen atom, a trifluoromethyl group, a lower alkyl group, a lower acyl group, NR⁴R^{4′}, a nitro group or a cyano group. Further, R represents a hydrogen atom, a lower alkyl group, a benzyl group or a lower acyl group which may have a substituent, and R⁴ and R^{4′} represents a hydrogen atom, a lower alkyl group, a lower acyl group, a benzyl group or SO₂R⁵, where R⁴ and R^{4′} may be same or different each other. R⁵ represents a lower alkyl group or a benzyl group. W represents an oxygen atom, a secondary nitrogen atom (NH) or a sulfur atom. * means an asymmetric carbon atom.], or a salt thereof.

A compound of the formula (II):

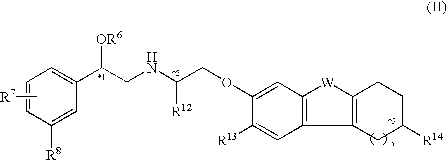

[wherein, R⁶ represents a hydrogen atom or a methyl group; R⁷ represents a hydrogen atom, a halogen atom, a hydroxyl group, a benzyloxy group, an amino group or a hydroxymethyl group; and R⁸ represents a hydrogen atom, a hydroxymethyl group, NHR⁹, SO₂NR¹⁰R^{10′} or a nitro group. Provided that R⁹ represents a hydrogen atom, a methyl group, SO₂R¹¹, a formyl group or CONHR^{12′} and R¹¹ represents a lower alkyl group, a benzyl group or NR¹R^{10′}. Further, R¹⁰ and R^{10′} represents a hydrogen atom, a lower alkyl group or a benzyl group, where R¹⁰ and R^{10′} may be same or different each other. R^{12′} represents a hydrogen atom or a lower alkyl group. Further, R¹² represents a hydrogen atom or a lower alkyl group. n represents 1 or 2, and W represents a secondary nitrogen atom, an oxygen atom or a sulfur atom. When n is 1, one of R¹³ and R¹⁴ represents a hydrogen atom and the other represents a hydrogen atom, an amino group, an acetylamino group or a hydroxyl group. When n represents 2, R¹⁴ represents a hydrogen atom and R¹³ represents a hydrogen atom, an amino group, an acetylamino group or a hydroxyl group. *1 represents an asymmetric carbon atom, and when neither R¹² nor R¹⁴ is a hydrogen atom, *2 and *3 mean an asymmetric carbon atom.], or a salt thereof.

A compound of the formula (III):

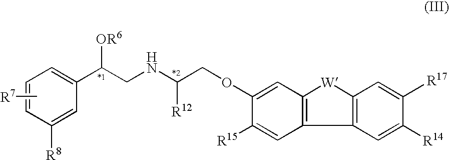

[wherein, R⁶ represents a hydrogen atom or a methyl group; R⁷ represents a hydrogen atom, a halogen atom, a hydroxyl group, a benzyloxy group, an amino group or a hydroxymethyl group; and R⁸ represents a hydrogen atom, a hydroxymethyl group, NHR⁹, SO₂NR¹⁰R^{10′} or a nitro group. Provided that R⁹ represents a hydrogen atom, a methyl group, SO₂R¹¹, a formyl group or CONHR^{12′}, and R¹¹ represents a lower alkyl group, a benzyl group or NR¹⁰R^{10′}. Further, R¹⁰ and R^{10′} represents a hydrogen atom, a lower alkyl group or a benzyl group, where R¹⁰ and R^{10′} may be same or different each other. R^{12′} represents a hydrogen atom or a lower alkyl group. Further, R¹² represents a hydrogen atom or a lower alkyl group. W′ represents a secondary nitrogen atom, an oxygen atom, a sulfur atom or a methylene group; and when W′ is a secondary nitrogen atom, an oxygen atom or a sulfur atom, R¹⁷ represents a hydrogen atom and one of R¹⁵ and R¹⁶ represents a hydrogen atom and the other represents a hydrogen atom, an amino group, an acetylamino group or a hydroxyl group. Further, when W′ is a methylene group, R¹⁵ and R¹⁶ each represent a hydrogen atom and R¹⁷ represents a hydrogen atom, an amino group, an acetylamino group or a hydroxyl group. *1 represents an asymmetric carbon atom, and when R¹² is a lower alkyl group *2 means an asymmetric carbon atom.], or a salt thereof.

The compound of the above general formula (I) and a pharmaceutically acceptable salt thereof are, for example, a novel compound prepared by a method described hereinafter. Further, the compound of general formula (II) and a pharmaceutically acceptable salt thereof are, for example, as described in WO99/01431. Further, the compound shown by general formula (III) and a pharmaceutically acceptable salt thereof are, for example, as described in Japanese Patent Application Laying-Open (kokai) No. 9-249623. In addition, as the pβ3-agonist of the present invention, any compound having a β3 agonist activity may be used, and examples include compounds described in U.S. Pat. No. 5,786,356 and WO98/43953.

In general formula (I), examples of a hydrogen atom include a fluorine atom, a chlorine atom, a bromine atom and an iodine atom, and of these a fluorine atom, a chlorine atom and a bromine atom are preferred. Further, the term “lower” of the term “lower alkyl group” means a straight or branched saturated hydrocarbon having 1-4 carbons, and examples thereof include methyl, ethyl, n-propyl, i-propyl, n-butyl, i-butyl, s-butyl and t-butyl. Further, the term “lower” of the term “lower acyl group” means a linear or branched acyl group having 1-6 carbons, and examples thereof include formyl, acetyl, propionyl, butyryl, isobutyryl, valeryl, isovaleryl, pivaloyl and hexanoyl.

R¹ represents a hydrogen atom, a halogen atom or a hydroxyl group, and preferred examples thereof include a hydrogen atom, a fluorine atom, a chlorine atom, a bromine atom and a hydroxyl group.

Further, R² represents a lower alkyl group or a benzyl group, and specific examples thereof include a methyl group, an ethyl group, a benzyl group, an n-propyl group, an i-propyl group, an n-butyl group, an i-butyl group, an s- butyl group and a t-butyl group, and, in particular, a methyl group and a benzyl group are preferred.

Further, R³ represents OR, a halogen atom, a trifluoromethyl group, a lower alkyl group, a lower acyl group, NR⁴R^{4′}, a nitro group or a cyano group (Provided, R means a hydrogen atom, a lower alkyl group, a benzyl group or a lower acyl group which may have a substituent. R⁴ and R^{4′} each independently represents a hydrogen atom, a lower alkyl group, a lower acyl group, a benzyl group or SO₂R⁵, where R⁴ and R^{4′} may be same or different. R⁵ represents a lower alkyl group or a benzyl group.), and of these OR is a preferred example. In addition, NR⁴R^{4′} is also a preferred example. A preferred example of R is a hydrogen atom, a lower alkyl group, or a lower acyl group which may have a substituent. More preferred examples for R⁴ and R^{4′} include a hydrogen atom, a lower acyl group or SO₂R⁵.

W represents an oxygen atom, a secondary nitrogen atom (NH) or a sulfur atom, and of these a secondary nitrogen atom is exemplified as a preferred example. As a substituent of a lower acyl group which may have a substituent, any substituent for a lower acyl group in a commercially available reagent may be used, and the substituent is not particularly limited, and an amino group that may be substituted with a lower alkyl group, a hydroxyl group or a lower alkoxy group are exemplified as preferred substituents.

The term “leaving group” described below means a chlorine atom, a bromine atom or an iodine atom, or a group that is eliminated such as a sulfonate such as a mesyl group or a tosyl group.

In general formula (I) described above, * means an asymmetric carbon atom, and it can be present as 2 enantiomers, R and S. Not only an optically pure isomer, but also a mixture of 2 isomers of an arbitrary ratio is included within the scope of present invention. From the viewpoint of expression of pharmacological activity, a preferred configuration of an ethanolamino chain asymmetric carbon * is absolute configuration R. Regarding an asymmetric carbon * of N-[3-[2-[2-(7-hydroxy-9H-carbazole-2-iloxy)ethylamino]-1-hydroxyethyl]phenyl]methanesulfonamide, in particular, a preferred example is a R-hydroxy form.

For the compounds of the present invention represented by general formula (I), preferred examples include a compound in which a combination of all substituents is [R¹ represents a hydrogen atom, a halogen atom or a hydroxyl group. R³ represents OR, a halogen atom, a trifluoromethyl group, a lower alkyl group, a lower acyl group, NR⁴R⁴, a nitro group or a cyano group. Further, R represents a hydrogen atom, a lower alkyl group, a benzyl group or a lower acyl group which may have a substituent, and R⁴ and R^{4′} each independently represents a hydrogen atom, a lower alkyl group, a lower acyl group, a benzyl group or SO₂R⁴, where R⁴ and R^{4′} may be same or different. R⁵ represents a lower alkyl group or a benzyl group.], or a salt thereof.

Further, in the present invention, preferred examples include a compound in which a combination of all substituents of general formula (I) described above is [R¹ represents a hydrogen atom, a halogen atom or a hydroxyl group. R³ represents OR, a halogen atom, a trifluoromethyl group, a lower alkyl group, NR⁴R^{4′}, a nitro group or a cyano group. Further, R represents a hydrogen atom, a lower alkyl group or a benzyl group, and R⁴ and R^{4′} each independently represents a hydrogen atom, a lower alkyl group or a benzyl group, where R⁴ and R^{4′} may be same or different.], or a salt thereof.

The compound of general formula (I) can, for example, be produced according to the following method.

(Production Method)

React a compound represented by general formula (IV)

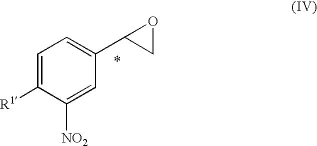

[wherein, R¹ represents a hydrogen atom, a halogen atom or a protected hydroxyl group, and * means an asymmetric carbon] with a compound represented by general formula (V)

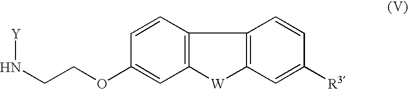

[wherein, W represents an oxygen atom, a secondary nitrogen atom (NH) or a sulfur atom. Y means a hydrogen atom or a protecting group for an amine, and R³ represents OR′, a halogen atom, a trifluoromethyl group, a lower alkyl group, a lower acyl group, NR⁴R^{4′}, a nitro group or a cyano group. Further, R′ represents a lower alkyl group, a lower acyl group which may have a substituent, a benzyl group or a protecting group for a hydroxyl group, and R⁴ and R^{4′} each independently represents a hydrogen atom, a lower alkyl group, a lower acyl group, a benzyl group, a protecting group for an amine or SO₂R⁵, where R⁴ and R^{4′} may be same or different. R⁵ represents a lower alkyl group or a benzyl group.], to form the compound represented by general formula (VI)

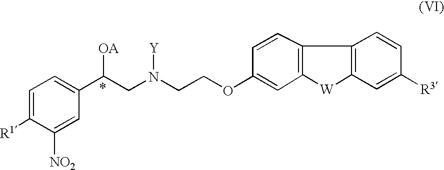

[wherein, A represents a hydrogen atom, and R^{1′}, R^{3′}, W, Y and * have the same meaning as described above, respectively.], and when Y is a hydrogen atom, convert it into a protecting group for an amine in advance, and then further reduce to form a compound represented by general formula (VII)

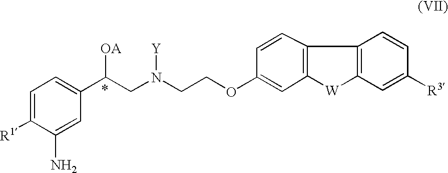

[wherein, Y represents a protecting group for an amine, and A, R^{1′}, R^{3′}, W and * have the same meaning as described above, respectively.]. Next, react the obtained compound with a compound represented by general formula (VIII)

XSO₂R² (VIII)

[wherein, R² represents a lower alkyl group or a benzyl group, and X represents a leaving group. A leaving group means a chlorine atom, a bromine atom or an iodine atom, or a group that is eliminated, such as a sulfonate such as a mesyl group or a tosyl group.] in the presence of alkali, to form a compound represented by general formula

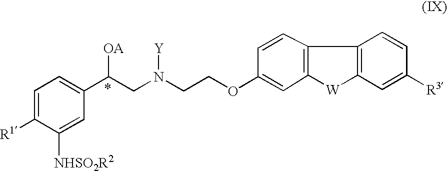

[wherein, A, R^{1′}, R², R^{3′}, W, Y and * have the same meaning as described above, respectively.], and when a protecting group is present at R^{1′}, R^{3′}and Y, by deprotecting these in one batch or in stages, a compound represented by general formula (I)

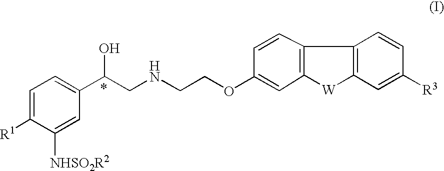

[wherein, R¹ represents a hydrogen atom, a halogen atom or a hydroxyl group, and R³ represents OR, a halogen atom, a trifluoromethyl group, a lower alkyl group, a lower acyl group, NR⁴R^{4′}, a nitro group or a cyano group. Further, R represents a hydrogen atom, a lower alkyl group, a benzyl group or a lower acyl group which may have a substituent, and R⁴ and R^{4′} each independently represents a hydrogen atom, a lower alkyl group, a lower acyl group, a benzyl group or SO₂R⁵, where R⁴ and R^{4′} may be same or different. R², R⁵, W and * have the same meaning as described above, respectively.] is obtained.

When including a protecting group for a hydroxyl group at R^{1′} or R^{3′}, any normally used group may be used as a protecting group for a hydroxyl group, and a protecting group is not particularly limited, and for example, as a protecting group which normally can be easily and selectively deprotected, a trialkylsilyl group, an alkoxyalkyl group and an acyl group are exemplified as preferred examples. When introducing or deprotecting these protecting groups for a hydroxyl group, a known method described in a book (for example, Greene, T. W., Wuts, P. G. M., et al. Protective Groups in Organic Synthesis, Wiley-Interscience Publication) can be used. For example, as exemplified by an example in which in the introduction of a t-butyl dimethyl silyl (TBDMS) group, a sililation reagent such as t-butyldimethylchlorosilane or t-butyldimethylsilyl-trifluoromethanesulfonate is allowed to act in alcohol in the presence of an acid scavenger. The added amount of sililation reagent is exemplified as normally around 1˜1.5×mol with respect to alcohol. Preferably, this reaction is normally carried out in an inactive medium. Examples of an inactive medium include dichloromethane, tetrahydrofuran, acetonitrile, pyridine and the like, and N,N-dimethylformamide is exemplified as a preferred example. A usage amount of an inactive medium is exemplified as around 1˜5 ml per 1 g of alcohol. Examples of an acid scavenger include triethylamine, N,N-diisopropyl ethylamine, pyridine and N,N-dimethylaminopyridine, and imidazole is exemplified as a preferred example. An added amount of the acid scavenger is exemplified as normally around 1˜3×mol with respect to alcohol. This reaction is normally carried out at −20˜80° C., and in particular, at 0° C. to room temperature is preferable, and for example, reaction for 1 to 5 hours is preferred.

Introduction of a benzyloxymethyl (BOM) group can be conducted by allowing chloromethyl-benzyl ether to act in alcohol in the presence of an acid scavenger. The added amount of chloromethyl-benzyl ether is exemplified as normally around 1˜1.5×mol with respect to alcohol. Preferably, this reaction is normally carried out in an inactive medium. Examples of an inactive medium include tetrahydrofuran, acetonitrile, N,N-dimethylformamide and the like, and dichloromethane is exemplified as a preferred example. A usage amount of an inactive medium is exemplified as around 1˜5 ml per 1 g of alcohol. Examples of an acid scavenger include triethylamine, pyridine and N,N-dimethylaminopyridine, and N,N-diisopropyl ethylamine is exemplified as a preferred example. An added amount of the acid scavenger is exemplified as normally around 1˜3×mol with respect to alcohol. This reaction is normally carried out at −20˜80° C., and in particular, at 0° C. to room temperature is preferable, and for example, reaction for 1 to 5 hours is preferred.

Further, introduction of an acetyl (Ac) group can be conducted by allowing an acetylating agent such as acetic anhydride or acetyl chloride to act in alcohol in the presence of an acid scavenger. The added amount of the acetylating agent is exemplified as normally around 1˜3×mol with respect to alcohol. Preferably, this reaction is normally carried out in an inactive medium. Preferred examples of an inactive medium include tetrahydrofuran, acetonitrile, dichloromethane, pyridine and the like. A usage amount of an inactive medium is exemplified as around 1˜5 ml per 1 g of alcohol. Preferred examples of an acid scavenger include triethylamine, N,N-diisopropyl ethylamine, pyridine, N,N-dimethylaminopyridine and the like. An added amount of the acid scavenger is exemplified as normally around 1˜3×mol with respect to alcohol. This reaction is normally carried out at −20˜80° C., and in particular, at 0° C. to room temperature is preferable, and for example, reaction for 1 to 5 hours is preferred.

Further, when including a protecting group for an amine at Y, R⁴, or R^{4′}, a protecting group is exemplified as an acyl group or an acyloxy group, or an easily deprotectable aralkyl group or the like. Examples of an easily deprotectable aralkyl group include a benzyl group, a substituted benzyl group, a naphthylmethyl group, a substituted naphthylmethyl group and the like, and a benzyl group is exemplified as a particularly preferred example. As an aralkyl group, for example, an aralkyl group having 7-16 carbons or the like can be used, and specific examples include a benzyl group, a phenethyl group, a 3-phenylpropyl group, a 4-phenylbutyl group or the like, as well as a (1-naphthyl)methyl group, a 2-(1-naphthyl)ethyl group, a 2-(2-naphthyl)ethyl group or the like, and on a phenyl group and a naphthyl group, for example, a suitable substituent such as an alkyl group, an alkoxy group or a halogen atom may be present in a suitable position. Introduction of these protecting groups may be conducted by a known method described in the aforementioned book.

The compound of general formula (VI) is a novel substance, and is characteristic as an important intermediate in synthesis of the compound represented by general formula (I). The compound of general formula (VI) can be obtained by reacting the compound represented by general formula (IV) and the compound represented by general formula (V) in a normal medium, for example, an alcohol organic solvent such as dimethylsulfoxide, straight chain or cyclic ether, dimethylformamide, dimethylacetamide or 2-butanol. While equimolar usage of the compound represented by general formula (IV) and the compound represented by general formula (V) is frequent, preferably the compound represented by general formula (V) is used in excess. A reaction temperature may be selected arbitrarily, and is exemplified as normally from room temperature to a reflux temperature of a selected solvent. A reaction tine can be arbitrarily selected according to the reaction conditions, and normally the reaction may be terminated at the time of maximum yield. Further, it is reported (Tetrahedron Lett., (1986), Vol. 27, p2451) that addition of trimethylsilylacetamide (TMSA), N,O-bis-(trimethylsilyl)acetamide, hexamethyidisilazane (HMDS) and bis-(trimethylsilyl)urea at time of reaction reduces the reaction time and increases the yield, and this method can be arbitrarily selected.

Further, the compound of general formula (VII) is also a novel substance, and is characteristic as an important intermediate in synthesis of the compound represented by general formula (I), and can be obtained by reducing the nitro group of general formula (VI) to form an amine (aniline). At the time of this reduction, in a case where substituent Y of general formula (VI) is a hydrogen atom, convert it into a protecting group for an amine in advance, and the reducing reaction can then be conducted, for example, in a solvent such as methanol, by hydrogenating in the presence of a platinum oxide catalyst or, alternatively, in the presence of iron powder or bivalent tin, by using hydrochloric acid or the like.

Further, the compound of general formula (IX) is also a novel substance, and is characteristic as an important intermediate in synthesis of the compound represented by general formula (I), and the compound represented by general formula (IX) can be obtained by converting various substituents at R² and conducting sulfonation of the amine (aniline) of the compound of general formula (VII) with the compound represented by general formula (VIII) using a method described in the literature (Kaiser C., et al., J. Med. Chem., (1974) Vol. 17, p.49). Further, of the protecting groups for a hydroxyl group or an amine at R^{1′}, R^{3′}, and Y, deprotect an existing protecting group by a method for deprotecting described hereunder to thereby obtain a compound represented by general formula (I).

Examples of the above sulfonation include reacting a known or commercially available compound of general formula (VIII) and a compound of general formula (VII) in a solvent such as pyridine at ice-cooling to room temperature. Deprotecting may be performed in sequence or in one batch, and preferably the sequence is such that a protecting group for a hydroxyl group at R^{1′} or R^{3′} is deprotected and then a protecting group for an amino group at Y or R^{3′} is deprotected. As the deprotecting conditions, a benzyl group that is a protecting group for a hydroxyl group at R^{1′} and R^{3′} can be deprotected by hydrogenolysis in a solvent such as methanol, using a catalyst such as palladium or nickel. Alternatively, a benzyl group or a methyl group or the like that is a protecting group for a hydroxyl group at R^{1′} or R^{3′} can be deprotected by treating with a Lewis acid such as boron tribromide in a solvent such as methylene chloride. Further, deprotection of an acetyl-protected hydroxyl group at R^{1′} and R^{3′} can be performed according to known conditions for hydrolysis of ester. Specific examples include a method in which, using alkali in alcohol, heating is performed at room temperature to the reflux temperature of the solvent. A triethylsilyl group or the like that is a protecting group for a hydroxyl group at R^{1′} or R^{3′} can be deprotected by treating in tetrahydrofuran containing acetic acid added with 3˜5×mol of tetrabutylammonium fluoride, at room temperature for 30 min to 5 hours. A benzyl group that is a protecting group for an amino group at Y or R^{3′} can be deprotected by hydrogenolysis in a solvent such as methanol, using a catalyst such as palladium or nickel. Further, in the case of an acetyl group being a protecting group for an amine at Y or R^{3′}, deprotection can be performed by treating with hydrochloric acid at room temperature in a solvent such as methanol, or by heating with alkali in water or a solvent such as methanol.

The compound represented by general formula (IV) is known, and a racemic body can be obtained, for example, by oxidation of a known corresponding styrene with an oxidizing agent such as m-chloroperbenzoic acid at 0° C. to room temperature in a solvent such as dichloromethane.

Furthermore, as an alternative method, a compound represented by general formula (X)

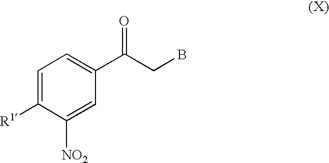

[wherein, R^{1′} represents the same meaning as described above, and B represents a chlorine atom, a bromine atom or an iodine atom.] is reduced by the method described below, or the like, to obtain general formula (XI)

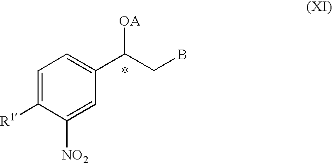

[wherein, R^{1′} and * represent the same meanings as described above, and A represents a hydrogen atom, and B represents a chlorine atom, a bromine atom or an iodine atom.], and when, as necessary, employing an iodine atom as substituent B, it can also be obtained by substituting iodine for a chlorine atom or a bromine atom and then epoxidizing by treating with alkali. That is, reduction of a compound represented by general formula (X), in a case where a * of a hydroxyl group of a compound represented by general formula (XI) is racemic, can be obtained by use of a reducing agent such as boron.

Further, regarding * in general formula (XI), when attempting to obtain an optical isomer of R or S, a chiral adjuvant such as shown by general formula (XII)

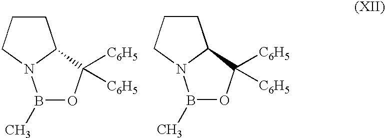

(where B represents a boron atom) may be used. That is, a compound represented by general formula (X) can be obtained by reducing with boron in the presence of the aforementioned chiral adjuvant. Preferably, the aforementioned reduction reaction is performed in a solvent such as tetrahydrofuran. Preparation of these chiral adjuvants and the reaction may be performed in accordance with a method described in the literature (E. J. Corey et al., J. Org. Chem., (1991), Vol. 56, p.442).

After reducing the compound represented by general formula (X) to obtain general formula (XI), in a case where substitution from a chlorine atom or bromine atom to an iodine atom is required, a method is exemplified in which the compound obtained by reducing as described above is further heated, in a solvent such as acetone, for 1 to 3 hours at reflux temperature with an iodination agent such as sodium iodide of 3˜10×mol with respect to the bromine body. Thereafter, by epoxidizing in the presence of an alkali such as sodium hydroxide solution of 1˜2 equivalence at 0° C. to room temperature, in a solvent such as methanol, the compound of general formula (IV) can be obtained. When obtaining general formula (IV) from general formula (XI), with regard to an asymmetric carbon *, the configuration thereof is retained. That is, an R body is obtained from an R body, and an S body is obtained from an S body.

The compound represented by general formula (X) is known, and a commercially available product can be utilized or the compound can be synthesized in accordance with a method described in the literature (for example, Larsen, A. A., et al. J. Med. Chem., (1967) Vol. 10, p.462; or Kaiser, C., et al. J. Med. Chem., (1974) Vol. 17, p.49).

On the other hand, the compound of general formula (V) is a novel substance and is characteristic as an important intermediate in synthesis of a compound represented by general formula (I).

The compound of general formula (V) can be obtained by reacting the compound of general formula (XIII)

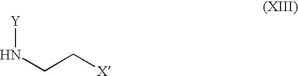

[wherein, Y means a protecting group for an amine, and X′ represents a chlorine atom, a bromine atom or a hydroxyl group] with the compound represented by general formula (XIV)

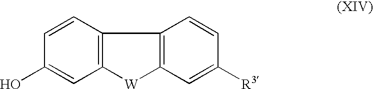

[wherein, W represents an oxygen atom, a secondary nitrogen atom (NH) or a sulfur atom. R^{3′} represents OR′, a halogen atom, a trifluoromethyl group, a lower alkyl group, a lower acyl group, NR⁴R^{4′}, a nitro group or a cyano group. Further, R′ represents a lower alkyl group, a lower acyl group which may have a substituent, a benzyl group or a protecting group for a hydroxyl group, and R⁴ and R^{4′} each independently represents a hydrogen atom, a lower alkyl group, a lower acyl group, a benzyl group, a protecting group for an amine or SO₂R⁵, where R⁴ and R^{4′} may be same or different. R⁵ represents a lower alkyl group or a benzyl group.]. Y, R⁴ or R^{4′} is a protecting group for an amine, and a protecting group for an amine is not particularly restricted as long as it is a group that is normally used, and examples include a benzyl group, a benzyloxycarbonyl group, a substituted benzyloxycarbonyl group or a t-butoxycarbonyl group, for which, normally, deprotection can be easily performed, or an acetyl group or trifluoroacetyl group or the like.

In a case where X′ is a chlorine atom or a bromine atom, reaction of the compounds of general formula (XIII) and general formula (XIV) may be conducted, for example, in an organic solvent, in the presence of a base, from room temperature to a reflux temperature of a selected solvent. Examples of a solvent include dimethylformamide, dimethylacetamide, acetonitrile, diglyme and tetrahydrofuran. As a base, use of potassium carbonate, sodium carbonate, sodium hydroxide, potassium hydroxide, triethylamine, pyridine, sodium hydride, sodium methoxide or the like, at 1˜10×mol with respect to general formula (XIV), is preferred.

In the case of a slow reaction, the compound of general formula (V) [Y means a protecting group for an amine] can be synthesized according to a method described in the literature (Bull. Chem. Soc. Jpn., (1982), Vol. 55, p.2504), or using a modified method thereof. For example, in dimethylformamide or acetonitrile, with respect to the compound of general formula (XIV), add 2˜5×mol of the compound of general formula (XIII), 5˜10×mol of 40% potassium fluoride-alumina, and as a modified method, further add potassium iodide of 0.1˜0.5 equivalent weight, and react at room temperature to 90° C.

In addition, by deprotecting protecting group Y for an amine, the amine compound of general formula (V) [Y means a hydrogen atom] can be obtained. In a case when deprotecting is for a benzyl group, deprotecting can be performed by, for example, in a solvent such as methanol, performing hydrogenolysis with palladium-carbon as a catalyst, or treating with hydrogen bromide-acetic acid. Further, in a case where protecting group Y is an acetyl group or a trifluoroacetyl group, by treating with alkali in a solvent such as methanol, the compound of general formula (V) [Y means a hydrogen atom] can be obtained.

Further, in a case where X′ in the compound of general formula (XIII) is a hydroxyl group, synthesis can be performed by conducting a Mitsunobu reaction with the compound of general formula (XIV). Specifically, reaction in a solvent such as tetrahydrofuran at 0° C. to room temperature, in the presence of triphenylphosphine of 1˜10 equivalent weight and azodicarboxylic acid diethyl of 1˜10 equivalent weight, is exemplified.

For the compound of general formula (XIII), first, using a commercially available amino alcohol, by protecting the amine with a protecting group Y a compound in which X′ is a hydroxyl group can be obtained. Next, a corresponding bromine body or iodine body can be synthesized by brominating or iodinating the hydroxyl group by a conventional method. Further, when Y is a benzyl group, it can be readily obtained by brominating a commercially available benzylaminoethanol, which is preferred. Further, if aminobromine body is easily available, it can be obtained by protecting the amine with protecting group Y. For example, under ice-cooling, react commercially available 2-bromoethylamine hydrobromide in methylene chloride in the presence of triethylamine with benzyloxycarbonyl chloride.

Further, the compound of general formula (V) can also be obtained by the following method. That is, react the compound of general formula (XIV) with the compound of general formula (XV)

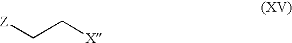

[wherein, Z represents a leaving group and X means a halogen atom. A leaving group means a chlorine atom, a bromine atom or an iodine atom, or a group that is eliminated, such as a sulfonate such as a mesyl group or a tosyl group.] to form the compound represented by general formula (XVI)

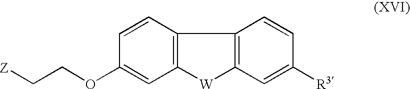

[wherein, W, Z and R^{3′} represent the same meaning as described above, respectively.]. Then, by substituting the compound represented by general formula (XVII)

YNH₂ (XVII)

[wherein, Y means a hydrogen atom or a protecting group for an amine.] for Z, the compound of general formula (V) can be obtained.

Of the compounds represented by general formula (XIV), when W is a secondary nitrogen atom, a compound other than a compound in which R^{3′} is a hydroxyl group, chloro or a methyl group is a novel compound, and it can be synthesized by the following method. Specifically, a compound in which R^{3′} is a hydroxyl group can be synthesized by a method described in the literature (Popri, S. P., Indian J. Chem. Sect. B, 1976, Vol 14B, p.371). By reacting this with alkyl halide in the presence of a base such as potassium carbonate, a compound can be synthesized in which arbitrary R^{3′} is OR′. Further, a protecting group can be introduced by the aforementioned method for introducing a protecting group. In addition, a compound in which R^{3′} is a bromine atom or a cyano group can be synthesized by deprotecting a compound described in the literature (Tidwell, R. R. et al. Eur. J. Med. Chem., 1997, Vol 32, p.781) in accordance with conventional conditions for deprotecting methyl ether. Further, a compound in which R^{3′} is a chlorine atom can be synthesized by deprotecting a compound described in the literature (Popri, S. P. et al. J. Med. Chem., 1976, Vol 16, p.425) in the same manner as described above. Further, a compound in which R^{3′} is a lower alkyl group can be synthesized by deprotecting, in the same manner as described above, a compound synthesized in accordance with a method described in the literature (Kapil, R. S. et al., Indian J. Chem. Sect. B, 1984, Vol 23B, p.296). Furthermore, as an alternative method, by performing coupling of the boron compound represented by general formula (XVIII)

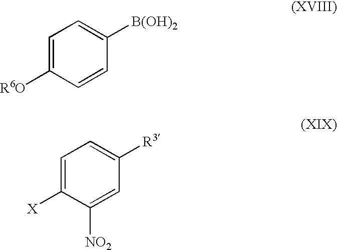

[wherein, R⁶ means a protecting group for a hydroxyl group. Further, B means a boron atom.] and the compound represented by general formula (XIX)

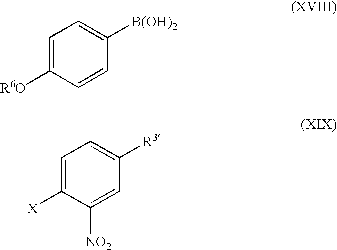

[wherein, X is a leaving group, and R^{3′} represents the same meaning as described above.] by means of a Suzuki reaction, the compound represented by general formula (XX)

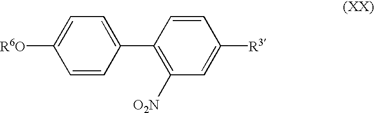

[wherein, R⁶ and R^{3′} represent the same meaning as described above, respectively.] can be formed. Then, by conducting a reductive cyclization reaction, the compound represented by general formula (XXI)

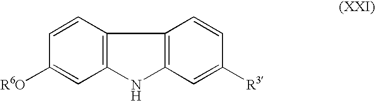

[wherein, R⁶ and R^{3′} represent the same meaning as described above, respectively.] is obtained, and by subsequently deprotecting R⁶, the compound of general formula (XIV) is obtained.

The compounds of general formula (XVIII) and general formula (XIX) can be obtained by procuring a commercially available product or by adding a protecting group to a commercially available product. Suzuki reaction may be conducted in accordance with a method described in a journal (Miyaura, Norio and Suzuki, Akira, YUKI GOSEI KAGAKU KYOUKAISHI, Vol. 46, 848 (1988)) or a method described in the literature (Holzapfel, C. W. et al. Heterocycles, Vol. 48, No. 8, 1513-18 (1998)).

The compound of general formula (XXI) can be synthesized by application of a method described in the literature (Cadogan, J. I. G., et al. J. Chem. Soc., 1965, 4831). Specifically, by heating the compound of general formula (XX) in the presence of trialkyl phosphite or triphenyl phosphite and conducting a reductive cyclization reaction, the carbazole derivative of general formula (XXI) can be obtained. As the phosphite used in the reaction, triethyl phosphite is preferred, and a usage amount is exemplified as 2˜10 equivalent weight, and use of 2˜4 equivalent weight is preferable. A reaction temperature is exemplified as from 80 to 180° C., and more preferably from 130 to 170° C. A reaction time is exemplified as from 1 to 24 hours, and more preferably from 3 to 10 hours. Thereafter, by selectively deprotecting R⁶ according to a conventional technique, the compound represented by general formula (XIV) can be obtained.

Further, for a compound of general formula (XIV), when W is an oxygen atom, a target compound can be obtained by deprotecting a methyl group of 3,7-dimethoxydibenzofuran described in the literature (Stransky, P. O. et al. J. Chem. Soc. Perkin Trans. 1, 1982, p.1605) according to a conventional method, and then protecting or realkylating one group only. Further, when W is a sulfur atom, a target compound can be obtained by reducing 3,7-dihydroxydibenzothiophene-5,5-dioxide described in the literature (Joullie, M. M. et al. J. Med. Chem., 1978, Vol. 21, p.1084) with lithium aluminium hydride to form 3,7-dihydroxydibenzothiophene, and then protecting or alkylating in the same manner as described above.

Further, as an alternative method, by reacting the compound represented by general formula (XI)

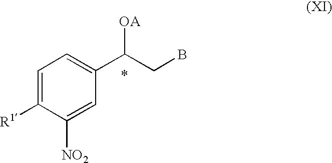

[wherein, A represents a protecting group for a hydroxyl group, and B represents a leaving group as described above. Further, R^{1′} and * represent the same meaning as described above, respectively.] with the compound represented by general formula (V)

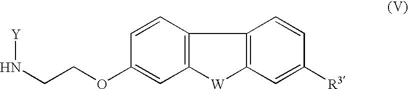

[wherein, Y represents a hydrogen atom or a protecting group for an amine, and W and R^{3′} represent the same meaning as described above, respectively.], the compound represented by general formula (VI) [wherein, A represents a protecting group for a hydroxyl group, and R^{1′}, R^{3′}, W, Y and * represent the same meaning as described above, respectively.] is obtained. Thereafter, the compound of general formula (I) can be obtained by the same method as described above.

Introduction and deprotection of protecting group A can be conducted in accordance with the method described above.

In addition, as a further alternative method, react the compound represented by general formula (X)

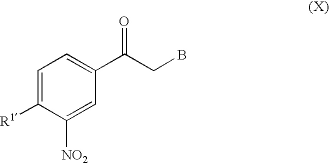

[wherein, R^{1′} represents the same meaning as described above. Also, B represents a leaving group as described above.] with the compound represented by general formula (V) [wherein, W, Y and R^{3′} represent the same meaning as described above, respectively.], to form the compound represented by general formula (XXII)

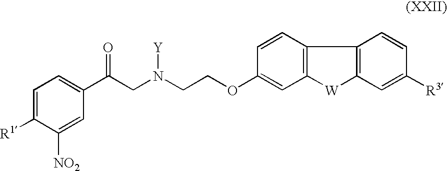

[wherein, R^{1′}, W, Y and R^{3′} represent the same meaning as described above, respectively.]. Then, by reducing the carbonyl group of this compound by the same method as described above, the compound of general formula (VI) [A represents a hydrogen atom] can be obtained. Thereafter, the compound of general formula (I) can be obtained by the same method as described above.

Reaction of the compound of general formula (X) with the compound of general formula (V) can be conducted by modification of a method described in the literature (Larsen, A. A. et al. J. Med. Chem., 1967, Vol. 10, p. 462), whereby, in a polar solvent such as acetonitrile, dimethylformamide, dimethylacetamide or dimethylsulfoxide, in the presence or absence of an amine as an acid trapping agent, reaction is conducted at ice-cooling to 60° C., and subsequently the carbonyl group is reduced with a reducing agent such as sodium borohydride or sodium cyanoborohydride at ice-cooling to room temperature, and then the protecting group is deprotected. In order to obtain an optically active substance, optical resolution may be conducted by the method described hereinafter, or asymmetric reduction may be conducted together with a hydrogen donor compound in the presence of a catalyst described above, or in the presence of a known catalyst of asymmetric reduction, such as one described in various literature (for example, Achiwa, K. et al. Chem. Pharm. Bull., 1995, Vol. 43, p.748; or Noyori, R. et al. J. Am. Chem. Soc., 1996, Vol. 118, p.2521), at the time of reduction.

Further, as a still further alternative method, react the compound represented by general formula (XXIII)

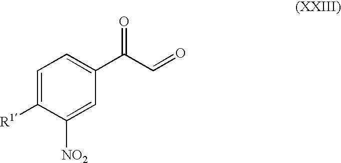

[wherein, R^{1′} represents the same meaning as described above.] with the compound represented by general formula (V) [wherein, Y represents a hydrogen atom, and W and R^{3′} represent the same meaning as described above, respectively.], and by further reducing the obtained Schiff base and carbonyl group, form the compound represented by general formula (VI) [wherein, A and Y represent a hydrogen atom, and R^{1′}, R^{3′} and W represent the same meaning as described above, respectively.]. Then after, as necessary, protecting A and Y in accordance with a conventional method, reduce a nitro base of this compound by the same method as described above, to thus obtain a compound of general formula (VII). Thereafter, the compound of general formula (I) can be obtained by the same method as described above.

This reaction is normally conducted in a medium, and is performed in the presence of a suitable reducing agent that can first further reduce the Schiff base obtained by condensation reaction and, at the same time, reduce the carbonyl group to a hydroxy group. Examples of the reducing agent include sodium borohydride, sodium cyanoborohydride and lithium cyanoborohydride. The amount of phenylglyoxal used is, with respect to the amine, 1-3×mol, and preferably 1-1.5×mol. As a reaction temperature, an arbitrary temperature can be selected, and it is exemplified as normally from room temperature to a reflux temperature of a selected solvent. A reaction time can be arbitrarily selected according to the reaction conditions, and normally reaction may be terminated at a time of maximum yield. For example, these reactions can be conducted in an alcohol solvent such as methanol or ethanol, preferably at a low temperature in the presence of sodium borohydride. To obtain an optically active substance, optical resolution may be conducted by a method described hereinafter.

The compound of general formula (XXIII) can be readily obtained by oxidizing, with an oxidizing agent such as selenium dioxide, an acetophenone substituted in R^{1′} in water or, for example, an organic solvent such as a cyclic ether such as dioxane or tetrahydrofuran. Further, as an alternative method, it can be produced in accordance with a method described in the literature (J. Am. Chem. Soc., 1957, Vol. 79. p. 6562).

Further, as a still further alternative method, react the amine represented by general formula (XXIV)

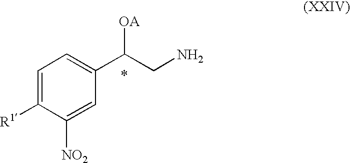

[wherein, A represents a protecting group for a hydroxyl group, and R^{1′} and * represent the same meaning as described above, respectively.], and the compound represented by general formula (XVI)

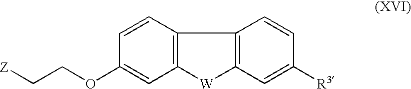

[wherein, W, R^{3′} and Z represent the same meaning as described above, respectively.] to obtain the compound represented by general formula (VI) [wherein, Y represents a hydrogen atom, A represents a protecting group for a hydroxyl group, and R^{1′}, R^{3′} and W represent the same meaning as described above, respectively.]. Then, after protecting the formed amine, the compound of general formula (I) can be synthesized by the same method as described above.

The coupling reaction with the amine is conducted in an organic solvent, if necessary in the presence of a proton receptor such as a tertiary amine (for example, triethylamine or the like), to thus obtain the compound of general formula (VI) [provided, Y represents a hydrogen atom]. The term “leaving group” means a chlorine atom, a bromine atom or an iodine atom, or a group that is eliminated at the time of the aforementioned reaction, such as a sulfonate such as a mesyl group or a tosyl group. As one example of a reaction condition, the usage amount of the amine of general formula (XXIV) is 1-10×mol with respect to the compound represented by general formula (XVI).

Since this reaction is slow, it is preferable to conduct it in an autoclave, and examples of a solvent to be used include alcohols such as methanol, ethanol or butanol, or a hydrogen halide such as methylene chloride or chloroform, tetrahydrofuran, dioxane or the like. A reaction temperature is, in general, 10˜150° C., and preferably 70-130° C. Reaction time is, in general, 5˜100 hours.

The compound of general formula (XXIV) can be obtained by hydrogenation of substituted mandelonitrile substituted at R^{1′}, for example, by reacting in the presence of a catalyst such as Raney nickel. Substituted mandelonitrile can be obtained as a racemic compound from reaction of substituted benzaldehyde and hydrogen cyanide or sodium cyanide and sodium hydrogen sulfite, and by forming a salt of an arbitrarily selected optically active acid and a diastereoisomer, in accordance with a normally performed method and technique, it can be readily isolated into an optically active isomer. Further, the optically active compound of general formula (XXIV) can be obtained by reacting optically active carboxylic acid obtained by hydrolyzing optically active substituted mandelonitrile with ammonia in the presence of a normally used condensing agent, and subsequently reducing.

The various compounds described in the present application, may, as necessary, be purified, which can be conducted using conventional known types of chromatography (column, flash column, thin layer, and high performance liquid), for example, employing the Rf value of the present specification as an index.

As mentioned above, the compound of general formula (I) can exist as 2 different optical isomers. Further, the compound of general formula (II) can exist as a maximum of 8 optical isomers, and the compound of general formula (III) can exist as a maximum of 4 optical isomers. The method of the present invention can provide both a pure optical isomer and a racemic mixture. The reactions described above do not change the relevant stereochemistry.

Accordingly, by starting from the compound of general formula (X) or (XXIII) which do not have an asymmetric carbon, or by starting from the compound of general formula (IV), (XI) or (XXIV) as a racemic compound, a racemic body can be obtained. Likewise, by starting from a pure optical isomer of the compound of general formula (IV), (XI) or (XXIV), for example an R isomer of general formula (IV), an R isomer only can be obtained. Further, when using an optically active isomer of the compound of general formula (IV), (XI) or (XXIV), a pure isomer can be obtained.

In a case where a mixture (racemic body) of 2 kinds of enantiomer is obtained, separation can be conducted by a suitable method such as fractional crystallization as a salt added with an optically active acid such as camphor sulfonic acid, mandelic acid or substituted mandelic acid. Fractional crystallization can be conducted using a suitable solvent, preferably a lower alkanol (for example, ethanol, isopropyl alcohol, or a mixture thereof).

Each group of enantiomer can be isolated into a pure isomer by formation of diastereomeric salt, chromatography using an optically active column, or another means. In a case where one of the starting materials is optically active, a diastereomeric mixture obtained in this manner is isolated into a pure isomer by the aforementioned technique. By isolating and purifying into an optically active isomer, since an isomer of higher activity only is used, enhancement of effects or dissociation of side effects or the like is possible, which is preferable for a medicament.

Preferably these compounds are, as necessary, added to a pharmaceutically acceptable carrier to form a medical compound useful as a medicament. Examples of a pharmaceutically acceptable carrier include an excipient, a bonding agent, such as carboxymethylcellulose, a disintegrating agent, a lubricant, an additive, and the like. When administering these compounds to humans, they can be administered orally in the form of a tablet, a powder, a granule, a capsule, a sugarcoated tablet, a solution, a syrup or the like. In addition, parenteral administration such as an injection is also possible. The dosage will differ according to the age, body weight, and degree of symptoms of a patient, however, in general, an amount of 0.01˜2000 mg is administered to one adult per day, and may be administered at one time or divided into several administrations. A dosing period generally consists of consecutive daily administration for several weeks to several months, however, the daily dosage and the dosing period can be increased or reduced depending on the symptoms of the patient. When concomitantly administering the therapeutic agent of the present invention, the substances may be mixed in one container, however, depending on the case, they may be stored in separate containers, and at the time of administration, administered to the same patient at substantially the same time.

“Urinary incontinence” is generally understood as “objectively verifiable involuntary urine leakage, which causes a hindrance in living respects and sanitary respects in daily life”, and “pollakiuria” is generally understood as “frequently having a micturition desire, and furthermore, feeling it is not possible to be patient before urination” (New Current 10(18), p2-7 (1999)). Obesity normally indicates a person whose BMI (Body Mass Index) is 25 or more, and it is reported that the ratio of people in this condition who develop hypertension, hyperlipemia or diabetes mellitus is high compared to people whose BMI is under 25 (Nature 404, p635-643 (2000)). Diabetes mellitus is classified into type I diabetes mellitus and type II diabetes mellitus, and in the present invention, type II diabetes mellitus is a more preferable object of treatment. Type II diabetes mellitus is characterized by insulin resistance and insulin hyposecretion. The gauge of diabetes mellitus is generally considered to be a fasting blood glucose level of 140 mg/dl or more, and a level of 200 mg/dl or more 2 hours after a 75 g glucose tolerance test (NIPPONRINSHO, Diabetes Mellitus 1, Vol. 55, p247-253 (1997)). Hyperlipemia consists of hypercholesterolemia and hypertriglyceridemia, and hypercholesterolemia, in particular, is a risk factor of arteriosclerosis. The diagnostic criteria of hyperlipemia is described (Molecular Medicine, Vol. 31, p544-550) as a serum cholesterol level of 220 mg/dl or more, a triglyceride level of 150 mg/dl or more, and a HDL (high density lipoprotein)-cholesterol level of 40 mg/dl or under.

In the present invention, an anticholinergic agent is an antagonist of a muscarinic receptor, and is as described in RINPI, Vol. 52, No. 5, p277-287 (1998), and specific examples include flavoxate hydrochloride, oxybutynin hydrochloride, propiverine hydrochloride, and tolterodine (Folia Pharmacologica Japonica, Vol. 113, p157-166 (1999); Eur. J. Pharmaco. 349, p285-292 (1998)). In particular, preferred examples are oxybutynin hydrochloride, propiverine hydrochloride, and tolterodine.

It is described in Medicine and Drug Journal, Vol. 36, No. 2, p151-157 (2000) that a monoamine reuptake inhibitor inhibits reuptake of serotonin and noradrenaline and increases monoamine concentration. Specific examples thereof include sibutramine, milnacipran, duloxetine, and venlafaxine, and in particular, sibutramine is a preferred example.

It is described in IYAKU JOURNAL, Vol. 36, No. 2, p137-140 (2000) that a selective serotonin reuptake inhibitor (SSRI) is capable of inhibiting only reuptake of serotonin, and is not capable of reuptake of noradrenaline or dopamine. Specific examples thereof include fluoxetine, sertraline, paroxetine, and fluvoxamine.

Further, it is described in J. Clin. Pharmacology 37, p453-473 (1997) that a lipase inhibitor is an inhibitor of lipase of the stomach or pancreas, and inhibits decomposition of triglyceride in food. In particular, orlistat is a preferred example thereof.

It is described in IGAKU NO AYUMI, Diabetes Mellitus 188, p309-313 (1999) that an insulin secretagogue promotes insulin secretion from β cells by stimulating K_{ATP} channels of pancreatic β-cells. Specific examples thereof include glibenclamide, glipizide, gliclazide, glimepiride, tolazamide, tolbutamide, acetohexamide, chlorpropamide, glyclopyramide, meglitinide, repaglinide, nateglinide and mitiglinide, and in particular, repaglinide, nateglinide and mitiglinide are preferred examples.

Examples of a Biguanide Include Metformin, and Buformin

It is described in IGAKU NO AYUMI, Diabetes Mellitus 188, p496-499 (1999) that an α-glucosidase inhibitor prevents absorption of sugar from the ciliated epithelium by inhibiting α-glucosidase of the small intestine. Specific examples thereof include acarbose, voglibose, miglitol and emiglitate, and in particular, acarbose and voglibose are preferred examples.

An insulin resistance improving agent is a medicament for ameliorating insulin resistance, which is one of the causes of type II diabetes mellitus, and at the present time a PPAR γ-agonist corresponds to this (Diabetes Mellitus 188, p500-503 (1999)). Specific examples thereof include troglitazone, pioglitazone, rosiglitazone, MCC-555, GI-262570, JTT-501 and KRP-297, and in particular, pioglitazone, rosiglitazone, MCC-555, GI-262570, JTT-501, and KPP-297 are preferred examples.

An HMG-CoA reductase inhibitor is an inhibitor of HMG-CoA reductase, a rate-limiting enzyme of the cholesterol biosynthetic pathway, and it leads to a reduction in blood cholesterol (Mol. Med. 31, p544-549 (1994)). Specific examples thereof include pravastatin, simvastatin, fluvastatin, atorvastatin, cerivastatin, nisvastatin and S-4522.

It is described in KISO TO RINSHOU (The clinical report), Vol. 16, p150-169 (1982) that an anion exchange resin binds to bile acid at a basic anion exchange resin and is excreted in feces, and specific examples thereof include cholestyramine and colestimide.

Clofibrate-base drugs have PPAR a agonistic activity and promote fatty acid oxidation (Mol. Med. 37, p83-90 (2000)). Specific examples thereof include clofibrate, simfibrate, clinofibrate, bezafibrate, fenofibrate, ciprofibrate and gemfibrozil.

Specific examples of nicotinic acid type drugs include nicotinic acid, nicomol, niceritrol and tocopherol nicotinate.

The therapeutic agent of the present invention has low toxicity, and therefore a larger amount thereof can be administered. Employing as an index the ratios of beneficial effect (for example, effective dose of β3 activity or the like) in each application with various toxicities, comparison of the indexes for use of a β3 agonist by itself and of each drug by itself, and for combined use of a β3 agonist and each drug showed that, by combined use in the present invention each index at the time of treatment was high, and the safety and high level of effectiveness were readily understood.

Examples of a selectable index of toxicity include mouth dryness, which is cited as a side effect of anticholinergic agents. Such mouth dryness can be measured by evaluating suppression of salivation induced with pilocarpine in accordance with the method of Nagao et al. (Folia Pharmacologica Japonica, Vol. 113, p156-166 (1999)). Further, elevation of blood pressure that is cited as a side effect of sibutramine can also be employed as an index of toxicity. Such elevation of blood pressure can be determined through use of a pressure transducer. Gastrointestinal dysfunction (fatty stool, defecation increase, abdominal pain, impending defecation desire) that is cited as a side effect of orlistat can also be employed as an index of toxicity. Such gastrointestinal dysfunction can be examined by analyzing the stool amount and the stool composition. Promotion of appetite that is cited as a side effect of insulin secretagogue can also be employed as an index of toxicity. Such promotion of appetite can be determined by observing body weight fluctuations using an animal, and hypoglycemia can be determined by investigating the concentration of glucose in blood. Loose passage that is cited as a side effect of α-glucosidase inhibitor can also be employed as an index of toxicity. Such loose passage and diarrhea can be investigated by, in the case of administration to animal, external observation of stool, or by analysis of stool amount and stool composition. Body weight gain that is cited as a side effect of insulin resistance improving agent can also be employed as an index of toxicity. Such body weight gain can be investigated by measurement of body weight after administration to animal. Gastrointestinal dysfunction that is cited as a side effect of HMG-CoA reductase inhibitor can also be employed as an index of toxicity. Such gastrointestinal dysfunction can be investigated by analysis of stool amount and stool composition. Gastrointestinal dysfunction that is cited as a side effect of clofibrate type drugs can also be employed as an index of toxicity. Such gastrointestinal dysfunction can be investigated by analysis of stool amount and stool composition.

### EXAMPLES

The present invention is further described in the following examples, however, the examples are not intended to limit the scope of the present invention. Compound a, which is a β3 agonist, used in these examples is (R)-N-[3-[2-[2-(7-hydroxy-9H-carbazole-2-iloxy)ethylamino]-1-hydroxyethyl]phenyl]methanesulfonamide. Hereinafter, similarly compound b is (R)-N-[5-[2-[2-(5,6,7,8-tetrahydro-9H-carbazole-2-iloxy)ethylamino]-1-hydroxyethyl]-2-hydroxyphenyl]methanesulfonamide, compound c is (R)-N-[5-[2-[2-(dibenzothiophene-3-iloxy)ethylamino]-1-hydroxyethyl]-2-hydroxyphenyl]methanesulfonamide, compound d is (R)-N-[5-[2-[2-(9H-carbazole-2-iloxy)ethylamino]-1-hydroxyethyl]-2-hydroxyphenyl]methanesulfonamide, compound e is (R)-N-[3-[2-[2-(9H-carbazole-2-iloxy)ethylamino]-1-hydroxyethyl]phenyl]methanesulfonamide, compound f is (R)-N′-[5-[2-[2-(9H-carbazole-2-iloxy)ethylamino]-1-hydroxyethyl]-2-hydroxyphenyl]-N,N-dimethyl sulfonyl amide, compound g is (R)-N′-[5-[2-[2-(dibenzofuran-3-iloxy)ethylamino]-1-hydroxyethyl]-2-hydroxyphenyl]-N,N-dimethyl sulfonyl amide, compound h is (R)-N-[5-[2-[2-(9H-carbazole-2-iloxy)ethylamino]-1-hydroxyethyl]-2-chlorophenyl]methanesulfonamide, and compound i is (R)-N-[5-[2-[2-(9H-carbazole-2-iloxy)ethylamino]-1-hydroxyethyl]-2-bromophenyl]methanesulfonamide.

Synthesis of Compound a

In accordance with a method described in the above-described production method, react a compound in which, in general formula (XIV), R^{3′} is a benzyloxy group and W is a secondary nitrogen atom with dibromoethane represented by general formula (XV), and further react with benzylamine to thereby obtain N-benzyl-2-(7-benzyloxy-9H-carbazole-2-iloxy)ethylamine represented by general formula (V). React this compound with a compound in which, in general formula (IV), R^{1′} is a hydrogen atom, and subsequently reduce the nitro group. Then, after mesylation compound a can be obtained by deprotecting the benzyl protecting group. Employ a hydrochloride according to a conventional method.

Rf=0.8 (chloroform/methanol=4/1 (free body));

Mass (m/e) 456 (MH+)

Further, compound b is disclosed in the specification of WO99/01431, and compounds c˜i are disclosed in the specification of Japanese Patent Application Laying-Open (kokai) No. 9-249623.

When examining the pharmacological effects for the above-described compounds a˜i, use a hydrochloride as these compounds. Such hydrochlorides can be prepared by a conventional method.

### EXAMPLE 1

### Dysuria Treatment Effect

Measurement of the contractive force of human urinary bladder compression muscle was conducted in accordance with the method of Takeda, M. et al. (J. Pharm. Exp. Ther. 288, p1367-1373 (1999). Specifically, contraction was provoked by carbachol (0.5×10⁻⁶ M), and a relaxation effect of compounds a, b, c, d, e, f, g, h, and i, and propiverine was investigated. Compounds a, b, c, d, e, f, g, h, i, propiverine, oxybutynin, and tolterodine significantly relaxed the bladder compression muscle at 10⁻⁷, 10⁻⁷, 10⁻⁸, 10⁻⁸, 10⁻⁶, 10⁻⁷, 10⁻⁷, 10⁻⁶, 10⁻⁶, 10⁻⁵, 10⁻⁸ and 10⁻⁸ M, respectively, however at 10⁻⁹, 10⁻⁹, 10⁻¹⁰, 10⁻¹⁰, 10⁻⁸, 10⁻⁹, 10⁻⁹, 10⁻⁸, 10⁻⁸, 10⁻⁶, 10⁻⁹ and 10⁻⁹ M, respectively, they did not relax the muscle significantly. When compounds consisting of a combination of 10⁻⁶ M of propiverine and each of 10⁻⁹ M of compound a, 10⁻⁹ M of compound b, 10⁻¹⁰ M of compound c, 10⁻¹⁰ M of compound d, 10⁻⁸ M of compound e, 10⁻⁹ M of compound f, 10⁻⁹M of compound g, 10⁻⁸ M of compound h, and 10⁻⁸ M compound i, respectively, were administered to the bladder compression muscle, the muscle was relaxed significantly in comparison with administration of each compound alone. Further, when compounds consisting of a combination of 10⁻⁹ M of oxybutynin and each of 10⁻⁹ M of compound a, 10⁻⁹ M of compound b, 10⁻¹⁰ M of compound c, 10⁻ M of compound d, 10⁻⁸ M of compound e, 10⁻⁹ M of compound f, 10⁻⁹ M of compound g, 10⁻⁸ M of compound h and 10⁻⁸ M of compound i, respectively, were administered to the bladder compression muscle, the muscle was relaxed significantly in comparison with administration of each compound alone. Further, when a compound consisting of a combination of 10⁻⁹ M of tolterodine and each of 10⁻⁹ M of compound a, 10⁻⁹ M of compound b, 10¹⁰ M of compound c, 10⁻¹⁰ M of compound d, 10⁻⁸ M of compound e, 10⁻⁹ M of compound f, 10⁻⁹ M of compound g, 10⁻⁸ M of compound h and 10⁻⁸ M of compound i, respectively, were administered to the bladder compression muscle, the muscle was relaxed significantly in comparison with administration of each compound alone.

Therefore, the therapeutic agent used in combination of the present invention exhibited a strong synergistic effect in therapeutic effects for pollakiuria and incontinence of urine, and it was confirmed that it is useful as a therapeutic agent for pollakiuria and incontinence of urine.

Regarding mouth dryness as a side effect often observed in administration of propiverine, oxybutynin, or tolterodine, measurement was conducted in accordance with the method of Nagao et al., (Folia Pharmacologica Japonica, Vol. 113, p156-166 (1999)), and suppression of salivation in the condition of a reduced dosage was observed.

### EXAMPLE 2

### Antiobestic Effect (I)

Mice induced with obesity by feeding with a high fat diet for 2 months were orally administered with sibutramine (SIB: 1 mg/kg), a β3 agonist (compound c: 0.3 mg/kg; compound d: 0.3 mg/kg; compound e: 3 mg/kg; compound g: 3 mg/kg; compound h: 1 mg/kg; compound i: 1 mg/kg), or SIB and a β3 agonist (compound c, compound d, compound e, compound g, compound h, and compound i) in combination (each dosage was the same as the aforementioned) for 2 weeks and bred, and body weight was measured. For the groups administered solely with SIB, compound c, compound d, compound e, compound g, compound h, or compound i, respectively, almost no change in body weight was observed. In contrast, for the groups administered with a combination of SIB and compound c, SIB and compound d, SIB and compound e, SIB and compound g, SIB and compound h, or SIB and compound i, respectively, a significant weight reduction was observed.

Therefore, it was confirmed that the therapeutic agent of the present case possesses a superior antiobestic effect.

Regarding elevation of blood pressure as a side effect of sibutramine, after administration of the drug, it was determined by use of a pressure transducer that elevation of blood pressure was alleviated to a greater extent.

### EXAMPLE 3

### Antiobestic Effect (II)

Mice induced with obesity by feeding with a high fat diet for 2 months were orally administered with orlistat (1 mg/kg), a β3 agonist (compound c: 0.3 mg/kg; compound d: 0.3 mg/kg; compound e: 3 mg/kg; compound g: 3 mg/kg; compound h: 1 mg/kg; compound i: 1 mg/kg), or orlistat and a β3 agonist (compound c, compound d, compound e, compound g, compound h, and compound i) in combination (each dosage was the same as the aforementioned) for 2 weeks and bred, and body weight was measured. For the groups administered solely with orlistat, compound c, compound d, compound e, compound g, compound h, or compound i, respectively, almost no change in body weight was observed. In contrast, for the groups administered with a combination of orlistat and compound c, orlistat and compound d, orlistat and compound e, orlistat and compound g, orlistat and compound h, or orlistat and compound i, respectively, a weight reduction was observed.

Therefore, it was confirmed that the therapeutic agent of the present case possesses a superior antiobestic effect

Gastrointestinal dysfunction (fatty stool, defecation increase, abdominal pain, impending defecation desire) as a side effect of orlistat, can be investigated, after administration of the drug, by analyzing stool amount and stool composition.

### EXAMPLE 4

### Antidiabetic Effect (II)

Mice were administered with insulin (INS: 0.2 U/kg, intraperitoneal injection), a β3 agonist (oral administration of compound a: 10 mg/kg; compound c: 0.3 mg/kg; compound d: 0.3 mg/kg; compound e: 3 mg/kg; compound g: 3 mg/kg; compound h: 1 mg/kg; compound i: 1 mg/kg, respectively), or INS and a β3 agonist (compound a, compound c, compound d, compound e, compound g, compound h, and compound i) in combination (each dosage and administration route was the same as the aforementioned), blood was collected after 30 min, and the amount of glucose contained in blood plasma was measured. The results showed that for the groups administered only with INS, compound a, compound c, compound d, compound e, compound g, compound h, or compound i, respectively, no change in blood glucose level was observed. In contrast, for the groups administered with a combination of INS and compound a, INS and compound c, INS and compound d, INS and compound e, INS and compound g, INS and compound h, or INS and compound i, respectively, the blood glucose level decreased significantly.

Therefore, it was confirmed that the therapeutic agent of the present case possesses a superior antidiabetic effect.

### EXAMPLE 5

### Antidiabetic Effect (II)

Mice fasted overnight were orally administered with glibenclamide (GLI: 10 mg/kg), a β3 agonist (compound a: 10 mg/kg; compound c: 0.3 mg/kg; compound d: 0.3 mg/kg; compound e: 3 mg/kg; compound g: 3 mg/kg; compound h: 1 mg/kg; compound i: 1 mg/kg), or GLI and a β3 agonist (compound a, compound c, compound d, compound e, compound g, compound h, and compound i) in combination (each dosage was the same as the aforementioned), and immediately glucose (1 g/kg, subcutaneous injection) was loaded. Blood was collected after 1 hour, and the amount of glucose contained in blood plasma was measured. In the groups administered solely with GLI, compound a, compound c, compound d, compound e, compound g, compound h, or compound i, there was almost no difference in the blood glucose level compared to mice not administered with a compound and for which only glucose loading was carried out. In contrast, in the groups administered with a combination of GLI and compound a, GLI and compound c, GLI and compound d, GLI and compound e, GLI and compound g, GLI and compound h, or GLI and compound i, respectively, the blood glucose level decreased significantly.

Therefore, it was confirmed that the therapeutic agent of the present case possesses a superior antidiabetic effect.

In this combined use, hypoglycemia, which is a side effect of glibenclamide, was not observed.

### EXAMPLE 6

### Antidiabetic Effect (III)

Genetically diabetic mice were orally administered with rosiglitazone (ROS: 1 mg/kg), a β3 agonist (compound c: 0.3 mg/kg; compound d: 0.3 mg/kg; compound e: 3 mg/kg; compound g: 3 mg/kg; compound h: 1 mg/kg; compound i: 1 mg/kg), or ROS and a β3 agonist (compound c, compound d, compound e, compound g, compound h, and compound i) in combination (each dosage was the same as the aforementioned) for 2 weeks. Blood was collected thereafter, and the amount of glucose contained in blood plasma was measured. For the groups administered solely with ROS, compound c, compound d, compound e, compound g, compound h, or compound i, no difference in the blood glucose level was observed. In contrast, in the groups administered with a combination of ROS and compound c, ROS and compound d, ROS and compound e, ROS and compound g, ROS and compound h, or ROS and compound i, respectively, the blood glucose level decreased significantly.

Therefore, it was confirmed that the therapeutic agent of the present case possesses a superior antidiabetic effect.

Further, in comparison with the group administered only ROS, weight reduction was observed in the groups administered with ROS and a compound in combination, and thus it was found that weight gain that is a side effect of ROS administration disappeared.

### EXAMPLE 7

### Antidiabetic Effect (IV)

Genetically diabetic mice were orally administered with acarbose (ACA: 1 mg/kg), a β3 agonist (compound c: 0.3 mg/kg; compound d: 0.3 mg/kg; compound e: 3 mg/kg; compound g: 3 mg/kg; compound h: 1 mg/kg; compound i: 1 mg/kg), or ACA and a β3 agonist (compound c, compound d, compound e, compound g, compound h, and compound i) in combination (each dosage was the same as the aforementioned) for 2 weeks. Blood was collected thereafter, and the amount of glucose contained in blood plasma was measured. For the groups administered solely with ACA, compound c, compound d, compound e, compound g, compound h, or compound i, respectively, no difference was observed in the blood glucose level. In contrast, in the groups administered with a combination of ACA and compound c, ACA and compound d, ACA and compound e, ACA and compound g, ACA and compound h, or ACA and compound i, respectively, the blood glucose level decreased.

Accordingly, it was confirmed that the therapeutic agent of this case possesses an excellent antidiabetic effect.

### EXAMPLE 8

### Antilipemic Effect (I)

By testing using a vehicle dog, it is possible to observe enhancement of antilipemic effect by combined use of pravastatin (PRA) and a β3 agonist. For example, according to the method of Tsujita, T. et al. (Biochim. Biophys. Acta, 877, p50-60 (1986)), it is shown that by administration to a vehicle dog of PRA: 0.625˜1.25 mg/kg, the blood cholesterol level and level of triglyceride in blood decrease. Further, since it is shown that a β3 agonist acts in a vehicle dog (KACHIKU SEIKAGAKU, Vol. 33, p13-18 (1996)), it is expected that compound a, b, c, d, e, f, g, h, and i also act therein. By combined use of pravastatin and a β3 agonist, which have different action mechanisms to each other, a synergistic decrease in the blood cholesterol level and level of triglyceride in blood can be acknowledged.

Accordingly, it was confirmed that the therapeutic agent of this case possesses an excellent antilipemic effect.

### EXAMPLE 9

### Antilipemic Effect (II)

Mice were orally administered with bezafibrate (BEZ: 3 mg/kg), a β3 agonist (compound c: 0.3 mg/kg; compound d: 0.3 mg/kg; compound e: 3 mg/kg; compound g: 3 mg/kg; compound h: 1 mg/kg; compound i: 1 mg/kg), or BEZ and a β3 agonist (compound c, compound d, compound e, compound g, compound h, and compound i) in combination (each dosage was the same as the aforementioned) for 2 weeks. Blood was collected thereafter, and the amount of triglyceride contained in blood plasma was measured. For the groups administered solely with BEZ, compound c, compound d, compound e, compound g, compound h, or compound i, respectively, no difference was observed in the blood glucose level. In contrast, for the groups administered with a combination of BEZ and compound c, BEZ and compound d, BEZ and compound e, BEZ and compound g, BEZ and compound h, or BEZ and compound i, respectively, triglyceride decreased.

Accordingly, it is expected that the therapeutic agent of this case possesses an excellent antilipemic effect.

All publications, patents and patent applications cited herein are incorporated herein by reference in their entirety.

### INDUSTRIAL APPLICABILITY

The present invention provides a therapeutic agent comprising at least one member selected from the group consisting of an anticholinergic agent, a monoamine reuptake inhibitor, a lipase inhibitor, a selective serotonin reuptake inhibitor, insulin, an insulin secretagogue, biguanide, an α-glucosidase inhibitor, an insulin resistance improving agent, a HMG-CoA reductase inhibitor, an anion exchange resin, a clofibrate type drug and a nicotinic acid type drug, and a compound having a β3 agonist activity. The therapeutic agent of the present invention has a dysuria treatment effect, an antiobestic effect, an antidiabetic effect, and an antilipemic effect.

What is claimed is:

1. A therapeutic agent comprising at least one member selected from the group consisting of an anticholinergic agent, a monoamine reuptake inhibitor, a lipase inhibitor, a selective serotonin reuptake inhibitor, insulin, an insulin secretagogue, biguanide, an α-glucosidase inhibitor, an insulin resistance improving agent, a HMG-CoA reductase inhibitor, an anion exchange resin, a clofibrate type drug and a nicotinic acid type drug, and a compound having a β3 agonist activity.

2. The therapeutic agent of claim 1, wherein the compound having a β3 agonist activity is a compound represented by any one of the following general formula (I), general formula (II), general formula (III) and a salt thereof.
A compound of the formula (I): 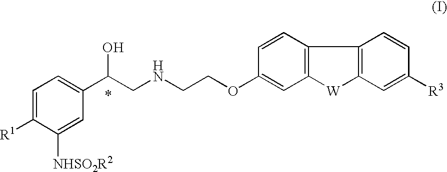
[wherein, R¹ represents a hydrogen atom, a halogen atom or a hydroxyl group, and R² represents a lower alkyl group or a benzyl group. R³ represents OR, a halogen atom, a trifluoromethyl group, a lower alkyl group, a lower acyl group, NR⁴R^{4′}, a nitro group or a cyano group. Further, R represents a hydrogen atom, a lower alkyl group, a benzyl group or a lower acyl group which may have a substituent, and R⁴ and R^{4′} represents a hydrogen atom, a lower alkyl group, a lower acyl group, a benzyl group or SO₂R⁵, where R⁴ and R^{4′} may be same or different each other. R⁵ represents a lower alkyl group or a benzyl group. W represents an oxygen atom, a secondary nitrogen atom (NH) or a sulfur atom. * means an asymmetric carbon atom. ], or a salt thereof.
A compound of the formula (II): 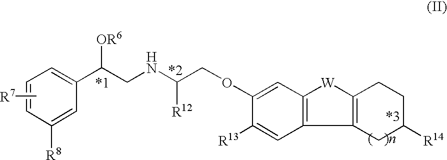
[wherein, R⁶ represents a hydrogen atom or a methyl group; R⁷ represents a hydrogen atom, a halogen atom, a hydroxyl group, a benzyloxy group, an amino group or a hydroxymethyl group; and R⁸ represents a hydrogen atom, a hydroxymethyl group, NHR⁹, SO₂NR¹⁰R^{10′} or a nitro group. Provided that R⁹ represents a hydrogen atom, a methyl group, SO₂R¹¹, a formyl group or CONHR^{12′}, and R¹¹ represents a lower alkyl group, a benzyl group or NR¹⁰R^{10′}. Further, R¹⁰ and R^{10′} represents a hydrogen atom, a lower alkyl group or a benzyl group, where R¹⁰ and R^{10′} may be same or different each other. R^{12′} represents a hydrogen atom or a lower alkyl group. Further, R¹² represents a hydrogen atom or a lower alkyl group. n represents 1 or 2, and W represents a secondary nitrogen atom, an oxygen atom or a sulfur atom. When n is 1, one of R¹³ and R¹⁴ represents a hydrogen atom and the other represents a hydrogen atom, an amino group, an acetylamino group or a hydroxyl group. When n represents 2, R¹⁴ represents a hydrogen atom and R¹³ represents a hydrogen atom, an amino group, an acetylamino group or a hydroxyl group. *1 represents an asymmetric carbon atom, and when neither R¹² nor R¹⁴ is a hydrogen atom, *2 and *3 mean an asymmetric carbon atom.], or a salt thereof.
A compound of the formula (III): 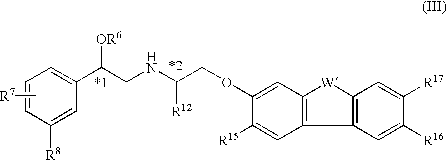
[wherein, R⁶ represents a hydrogen atom or a methyl group; R⁷ represents a hydrogen atom, a halogen atom, a hydroxyl group, a benzyloxy group, an amino group or a hydroxymethyl group; and R⁸ represents a hydrogen atom, a hydroxymethyl group, NHR⁹, SO₂NR¹⁰R^{10′} or a nitro group. Provided that R⁹ represents a hydrogen atom, a methyl group, SO₂R¹¹, a formyl group or CONHR ^{12′}, and R¹¹ represents a lower alkyl group, a benzyl group or NR¹⁰R^{10′}. Further, R¹⁰ and R^{10′} represents a hydrogen atom, a lower alkyl group or a benzyl group, where R¹⁰and R^{10′} may be same or different each other. R^{12′} represents a hydrogen atom or a lower alkyl group. Further, R¹² represents a hydrogen atom or a lower alkyl group. W′ represents a secondary nitrogen atom, an oxygen atom, a sulfur atom or a methylene group; and when W′ is a secondary nitrogen atom, an oxygen atom or a sulfur atom, R¹⁷ represents a hydrogen atom and one of R¹⁵ and R¹⁶ represents a hydrogen atom and the other represents a hydrogen atom, an amino group, an acetylamino group or a hydroxyl group. Further, when W′ is a methylene group, R¹⁵ and R¹⁶ each represent a hydrogen atom and R¹⁷ represents a hydrogen atom, an amino group, an acetylamino group or a hydroxyl group. *1 represents an asymmetric carbon atom, and when R¹² is a lower alkyl group *2 means an asymmetric carbon atom.], or a salt thereof.

3. The therapeutic agent of claim 1 or 2, which is a therapeutic agent for pollakiuria and incontinence of urine, comprising at least an anticholinergic agent and a compound having a β3 agonist activity.

4. The therapeutic agent of claim 3, wherein the anticholinergic agent is any one of flavoxate hydrochloride, oxybutynin hydrochloride, propiverine hydrochloride and tolterodine.

5. The therapeutic agent of claim 3, wherein the anticholinergic agent is any one of oxybutynin hydrochloride, propiverine hydrochloride and tolterodine.

6. The therapeutic agent of claim 1 or 2, which is a therapeutic agent for obesity, comprising at least one member selected from the group consisting of a monoamine reuptake inhibitor, a lipase inhibitor and a selective serotonin reuptake inhibitor, and a compound having a β3 agonist activity.

7. The therapeutic agent of claim 6, comprising at least a monoamine reuptake inhibitor and a compound having a β3 agonist activity.

8. The therapeutic agent of claim 6 or 7, wherein the monoamine reuptake inhibitor is any one of sibutramine, milnacipran, duloxetine and venlafaxine.

9. The therapeutic agent of claim 6 or 7, wherein monoamine reuptake inhibitor is sibutramine.

10. The therapeutic agent of claim 6, comprising at least a lipase inhibitor and a compound having a β3 agonist activity.

11. The therapeutic agent of claim 6 or 10, wherein the lipase inhibitor is orlistat.

12. The therapeutic agent of claim 6, comprising at least a selective serotonin reuptake inhibitor and a compound having a β3 agonist activity.

13. The therapeutic agent of claim 6 or 12, wherein the selective serotonin reuptake inhibitor is fluoxetine, sertraline, paroxetine or fluvoxamine.

14. The therapeutic agent of claim 1 or 2, which is a therapeutic agent for type II diabetes mellitus, comprising at least one member selected from the group consisting of insulin, an insulin secretagogue, biguanide, an α-glucosidase inhibitor and an insulin resistance improving agent, and a compound having a β3 agonist activity.

15. The therapeutic agent of claim 14, comprising at least insulin and a compound having a β3 agonist activity.

16. The therapeutic agent of claim 14, comprising at least an insulin secretagogue and a compound having a β3 agonist activity.

17. The therapeutic agent of claim 14 or 16, wherein the insulin secretagogue is glibenclamide, glipizide, gliclazide, glimepiride, tolazamide, tolbutamide, acetohexamide, chlorpropamide, glyclopyramide, meglitinide, repaglinide, nateglinide or mitiglinide.

18. The therapeutic agent of claim 14 or 16, wherein the insulin secretagogue is glibenclamide.

19. The therapeutic agent of claim 14 or 16, wherein the insulin secretagogue is repaglinide, nateglinide or mitiglinide.

20. The therapeutic agent of claim 14, comprising at least biguanide and a compound having a β3 agonist activity.

21. The therapeutic agent of claim 14 or 20, wherein the biguanide is metformin or buformin.

22. The therapeutic agent of claim 14, comprising at least an α-glucosidase inhibitor and a compound having a β3 agonist activity.

23. The therapeutic agent of claim 14 or 22, wherein the α-glucosidase inhibitor is acarbose, voglibose, iniglitol or emiglitate.

24. The therapeutic agent of claim 14 or 22, wherein the α-glucosidase inhibitor is acarbose.

25. The therapeutic agent of claim 14, comprising at least an insulin resistance improving agent and a compound having a β3 agonist activity.

26. The therapeutic agent of claim 14 or 25, wherein the insulin resistance improving agent is troglitazone, pioglitazone, rosiglitazone, MCC-555, GI-262570, JTT-501 or KRP-297.

27. The therapeutic agent of claim 14 or 25, wherein the insulin resistance improving agent is rosiglitazone.

28. The therapeutic agent of claim 14 or 25, wherein the insulin resistance improving agent is MCC-555, GI-262570, JTT-501 or KRP-297.

29. The therapeutic agent of claim 1 or 2, which is a therapeutic agent for hyperlipemia, comprising at least one member selected from the group consisting of a HMG-CoA reductase inhibitor, an anion exchange resin, a clofibrate type drug and a nicotinic acid type drug, and a compound having a β3 agonist activity.

30. The therapeutic agent of claim 29, comprising at least a HMG-CoA reductase inhibitor and a compound having a β3 agonist activity.

31. The therapeutic agent of claim 29 or 30, wherein the HMG-CoA reductase inhibitor is pravastatin, simvastatin, fluvastatin, atorvastatin, cerivastatin, nisvastatin or S-4522.

32. The therapeutic agent of claim 29 or 30, wherein the HMG-CoA reductase inhibitor is pravastatin.

33. The therapeutic agent of claim 29 or 30, wherein the HMG-CoA reductase inhibitor is simvastatin, fluvastatin, atorvastatin, cerivastatin, nisvastatin or S-4522.

34. The therapeutic agent of claim 29, comprising at least an anion exchange resin and a compound having a β3 agonist activity.

35. The therapeutic agent of claim 29 or 34, wherein the anion exchange resin is cholestyramine or colestimide.

36. The therapeutic agent of claim 29, comprising at least a clofibrate type drug and a compound having a β3 agonist activity.

37. The therapeutic agent of claim 29 or 36, wherein the clofibrate type drug is clofibrate, simfibrate, clinofibrate, bezafibrate, fenofibrate, ciprofibrate or gemfibrozil.

38. The therapeutic agent of claim 29 or 36, wherein the clofibrate type drug is bezafibrate.

39. The therapeutic agent of claim 29, comprising at least a nicotinic acid type drug and a compound having a β3 agonist activity.

40. The therapeutic agent of claim 29 or 39, wherein the nicotinic acid type drug is nicotinic acid, nicomol, niceritrol or tocopherol nicotinate.

41. A treatment method which comprises administration of a therapeutic agent comprising at least one member selected from the group consisting of an anticholinergic agent, a monoamine reuptake inhibitor, a lipase inhibitor, a selective serotonin reuptake inhibitor, insulin, an insulin secretagogue, biguanide, an α-glucosidase inhibitor, an insulin resistance improving agent, a HMG-CoA reductase inhibitor, an anion exchange resin, a clofibrate type drug and a nicotinic acid type drug, and a compound having a β3 agonist activity.

42. The treatment method of claim 41, wherein the compound having a β3 agonist activity is a compound represented by any one of the following general formula (I), general formula (II), general formula (III) and a salt thereof.
A compound represented by the general formula (I): 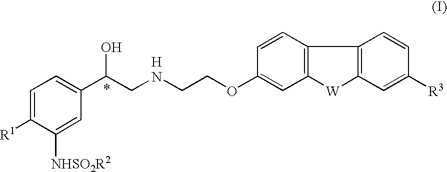
[wherein, R¹ represents a hydrogen atom, a halogen atom or a hydroxyl group, and R² represents a lower alkyl group or a benzyl group. R³ represents OR, a halogen atom, a trifluoromethyl group, a lower alkyl group, a lower acyl group, NR⁴R^{4′}, a nitro group or a cyano group. Further, R represents a hydrogen atom, a lower alkyl group, a benzyl group or a lower acyl group which may have a substituent, and R⁴ and R⁴ represents a hydrogen atom, a lower alkyl group, a lower acyl group, a benzyl group or SO₂R⁵, where R⁴ and R^{4′} may be same or different each other. R⁵ represents a lower alkyl group or a benzyl group. W represents an oxygen atom, a secondary nitrogen atom (NH) or a sulfur atom. * means an asymmetric carbon atom. ], or a salt thereof.
A compound represented by the general formula (II): 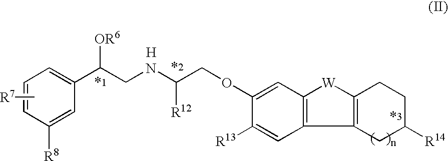
[wherein, R⁶ represents a hydrogen atom or a methyl group; R⁷ represents a hydrogen atom, a halogen atom, a hydroxyl group, a benzyloxy group, an amino group or a hydroxymethyl group; and R⁸ represents a hydrogen atom, a hydroxymethyl group, NHR⁹, SO₂NR¹⁰R^{10′} or a nitro group. Provided that R⁹ represents a hydrogen atom, a methyl group, SO₂R¹¹, a formyl group or CONHR^{12′}, and R¹¹ represents a lower alkyl group, a benzyl group or NR¹⁰R^{10′}. Further, R¹⁰ and R^{10′}, represents a hydrogen atom, a lower alkyl group or a benzyl group, where R¹⁰ and R^{10′} may be same or different each other. R^{12′} represents a hydrogen atom or a lower alkyl group. Further, R¹² represents a hydrogen atom or a lower alkyl group. n represents 1 or 2, and W represents a secondary nitrogen atom, an oxygen atom or a sulfur atom. When n is 1, one of R¹³ and R¹⁴ represents a hydrogen atom and the other represents a hydrogen atom, an amino group, an acetylamino group or a hydroxyl group. When n represents 2, R¹⁴ represents a hydrogen atom and R¹³ represents a hydrogen atom, an amino group, an acetylamino group or a hydroxyl group. * 1 represents an asymmetric carbon atom, and when neither R¹² nor R¹⁴ is a hydrogen atom, *2 and *3 mean an asymmetric carbon atom.], or a salt thereof.
A compound represented by the general formula (III): 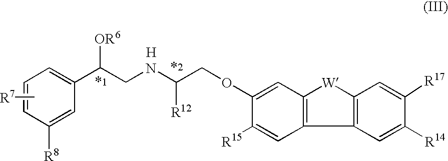
[wherein, R⁶ represents a hydrogen atom or a methyl group; R⁷ represents a hydrogen atom, a halogen atom, a hydroxyl group, a benzyloxy group, an amino group or a hydroxymethyl group; and R⁸ represents a hydrogen atom, a hydroxymethyl group, NHR⁹, SO₂NR¹⁰R^{10′} or a nitro group. Provided that R⁹ represents a hydrogen atom, a methyl group, SO₂R¹¹, a formyl group or CONHR^{12′} and R¹¹ represents a lower alkyl group, a benzyl group or NR¹⁰R^{10′}. Further, R¹⁰ and R^{10′} represents a hydrogen atom, a lower alkyl group or a benzyl group, where R¹⁰ and R^{10′} may be same or different each other. R^{12′} represents a hydrogen atom or a lower alkyl group. Further, R¹² represents a hydrogen atom or a lower alkyl group. W′ represents a secondary nitrogen atom, an oxygen atom, a sulfur atom or a methylene group; and when W′ is a secondary nitrogen atom, an oxygen atom or a sulfur atom, R¹⁷ represents a hydrogen atom and one of R¹⁵ and R¹⁶ represents a hydrogen atom and the other represents a hydrogen atom, an amino group, an acetylainino group or a hydroxyl group. Further, when W′ is a methylene group, R¹⁵ and R¹⁶ each independently represent a hydrogen atom and R¹⁷ represents a hydrogen atom, an amino group, an acetylamino group or a hydroxyl group. *1 represents an asymmetric carbon atom, and when R¹² is a lower alkyl group *2 means an asymmetric carbon atom.], or a salt thereof.

43. The treatment method of claim 41 or 42, which is a treatment method for pollakiuria and incontinence of urine, comprising administration of a therapeutic agent comprising at least an anticholinergic agent and a compound having a β3 agonist activity.

44. The treatment method of claim 41 or 42, which is a treatment method for obesity, comprising administration of a therapeutic agent comprising at least one member selected from the group consisting of a monoamine reuptake inhibitor, a lipase inhibitor and a selective serotonin reuptake inhibitor, and a compound having a β3 agonist activity.

45. The treatment method of claim 41 or 42, which is a treatment method for diabetes mellitus, comprising administration of a therapeutic agent comprising at least one member selected from the group consisting of insulin, an insulin secretagogue, biguanide, an α-glucosidase inhibitor and an insulin resistance improving agent, and a compound having a β3 agonist activity.

46. The treatment method of claim 41 or 42, which is a treatment method for hyperlipemia, comprising administration of a therapeutic agent comprising at least one member selected from the group consisting of a HMG-CoA reductase inhibitor, an anion exchange resin, a clofibrate type drug and a nicotinic acid type drug, and a compound having a β3 agonist activity.
